(19) United States
(12) Patent Application Publication
(10) Pub. No.: US 2022/0023469 A1
(43) Pub. Date: Jan. 27, 2022

(54) PORTABLE DEVICE FOR DISINFECTING GROOMING UTENSILS AND METHOD FOR TREATMENT OF HAIR STYLING UTENSILS

(71) Applicant: Gregory T. Hornback, Glen Ellyn, IL (US)

(72) Inventor: Gregory T. Hornback, Glen Ellyn, IL (US)

(21) Appl. No.: 17/385,729

(22) Filed: Jul. 26, 2021

Related U.S. Application Data
(60) Provisional application No. 63/056,547, filed on Jul. 24, 2020.

(51) Int. Cl. A61L 2/10; A61L 2/26

(52) U.S. Cl. CPC A61L 2/10; A61L 2/26; A61L 2202/122; A61L 2202/14; A61L 2202/11; A61L 2202/16

(57) ABSTRACT

The present invention relates to disinfection treatment for hair grooming utensils, such as a comb or shears used for styling and cutting hair. More specifically, the present invention relates to a hand-held portable device for sanitizing at least a portion of an elongated hair grooming utensil with the use of ultra violet light, wherein the elongated utensil is removably inserted into an opening of the housing of the device and into a narrow elongated inner chamber, and ultra-violet light emitting elements are positioned in close proximity to the utensil for sanitizing the outer surface of the utensil.

### PRIORITY CLAIM

This application claims priority to U.S. Provisional Patent Application No. 63/056,547, filed on Jul. 24, 2020, the contents of which are fully incorporated by reference herein.

### BACKGROUND

Grooming utensils, such as hair combs and shears, used by professionals are often costly and frequently used by a professional groomer during a given day. Typically, in additional to periodic cleaning and care of the utensils, professional stylists will sanitize combs in a disinfecting liquid. For example, Barbicide® is a solution with an active ingredient that acts as a germicide, fungicide and virucide. A stylist working in a salon often will have access to a container of such liquid disinfectant for periodically disinfecting utensils, such as by placing combs in the container for a period of time and dipping the blades of shears into the solution. Due to cost and space constraints, often a single container of such disinfecting solution is shared between multiple stylists in a salon. This is disadvantageous for multiple reasons. For example, at times, utensils may be mixed or accidentally taken by another stylist, and it is difficult or inconvenient for a stylist to walk away from a client to switch utensils, to place one in the container and take another out. Further, in a salon or barber shop, counter space is limited and needed for other equipment, making it less desirable to use such space for disinfectant containers.

Practically, because a stylist sometimes accidentally drop a hair styling utensil, such as a comb, when working with a client, there is a need to have immediate and convenient access to disinfecting treatment and/or to substitute a utensil being used for one already disinfected. Thus, there is a need for a stylist to have ready access to insert a utensil for treatment, and preferably to grasp an already disinfected utensil from the treatment container. Such accessibility is necessary because the need to swap utensils occurs while a stylist is in the midst of working. Traditionally, such disinfecting treatment involves containers of disinfecting solution, requiring multiple containers located conveniently for every stylist station in a salon, thus requiring multiple containers and taking up valuable counter space in the salon or barber shop. Further, there are times when it is impractical or undesirable for stylists to share equipment, due to a number of factors including the space needed for stylists to work, customer preferences, or in the event of a social distancing mandate to prevent spread of illness as may be required due to pandemic concerns.

Further, there are situations in which it is not practical for a stylist to use a container of disinfecting solution, such as when a professional stylist is working in locations other than at a salon. This situation arises out of convenience, to provide professional hair grooming at a person's home, or may arise out of necessity, such as when salons and barber shops are closed due to holidays or social distancing rules, etc. When this situation arises, a stylist must either bring a container of disinfecting solution or have multiple utensils, which are both undesirable options for a professional stylist or even for personal use of utensils.

Prior art disinfecting devices do not meet this need. Rather, common disinfecting devices, are not portable, such as in the case of a Barbicide® bath container, and other prior art devices are not suitable for use with hair grooming utensils. For example, while devices are available for sanitizing a small personally item, such as a toothbrush, keys or cell phone, such devices are not suitable for sanitizing the entire extent of an elongated surface of a comb or shears, are not typically suitable for portable use or easy access to the article being treated, and do not have features that will ensure treatment of the entire surface of an object. Such devices, for example as disclosed in U.S. Pat. No. 6,972,416 issued to Huang et al, or U.S. Publication No. 2012/0138491, which are not suitable for use to effectively sanitize hair grooming utensils such as combs or shears. Other devices, such as described in U.S. Publication No. 2006/0147339, have a lid that must be opened to insert the entire article to be treated—i.e., not allowing a protruding portion such as the handle of a comb or shears when in use, and not facilitating easy and rapid access to the object being treated. Such devices, if used for grooming utensils, would be bulky and inconvenient, due to the size of a container necessary for receiving the entirety of shears or combs, and because a user must open a lid to insert or remove the article being treated. Thus, such box-type devices do not provide a stylist easy access to a sanitized utensil while working, and are not convenient when needing to swap utensils while working with a customer. In addition, such devices potentially expose the UV element(s) within the device to dust that may compromise efficacy of the device, and are not conducive to providing adequate UV exposure of the ultra-violet light to the surfaces of the article being treated. Specifically, because the UV light-emitting lights of such devices are relatively distant from at least some of the outer surface of a utensil, such devices are ineffective and/or require an extended period of time for treatment. In sum, such devices suffer from the same setbacks of other common methods or products, as they do not offer portability, convenience or suitable comprehensive treatment of an elongated article such as grooming utensils, and do not allow for simple and rapid insertion and removal of the article being treated.

Hence, there is a need for an alternative means for disinfecting hair stylist utensils such as combs and shears, which is portable, avoids use of liquids or harsh chemicals, is compact, is efficient with a high level of efficacy and provides easy accessibility for exchanging utensils for treatment. The present invention solves these and other shortfalls of the prior art devices and methods.

### SUMMARY

The present invention relates to disinfection treatment for sanitizing hair grooming utensils, such as a comb or shears used for styling and cutting hair. More specifically, the present invention relates to a compact hand-held portable device for disinfecting hair grooming utensils with the use of ultra violet light, which is particularly useful for a hair stylist performing grooming services, whether in a salon or at a remote location such as may be needed for a hair stylist using grooming utensils at a person's home or similar location away from a traditional salon setting. A device embodying features of the present invention allows for portability, is compact, provides rapid disinfection of the entire surface of a region of the grooming utensil and allows utensils to be easily and rapidly inserted and removed by a user. More specifically, the present invention provides a portable sanitation device for sanitizing treatment of hair grooming utensils that comprises a housing formed of an outer sidewall arrangement and has an inner sidewall arrangement at least partially defining an inner chamber. The housing has an opening that is configured to allow passage of a grooming utensil into the inner chamber without the need to open a lid or cover. The inner sidewall arrangement has at least two substantially opposed sidewalls extending along an extent of the elongated inner chamber, and a plurality of opposed ultra-violet emitting elements are positioned on the opposed sidewalls. The disinfecting elements are configured to emit ultra-violet (UV) light toward the inner chamber for sanitization, i.e., disinfection treatment of the utensil when positioned within the inner chamber, and the utensil is readily removed by a user through the opening. In accordance with an aspect of the present invention, an array of LED elements are provided on the inner sidewall in close proximity to the surface of the utensil for treatment. This aspect of the device disclosed herein provides a high degree of efficacy for disinfecting utensils and rapid treatment time.

### BRIEF DESCRIPTION OF THE DRAWINGS

FIG. 1 is an elevated perspective view of a portable disinfection device for grooming utensils according to an embodiment of the present invention, shown in use with grooming shears inserted into the device;

FIG. 2 is an elevated perspective view of a portable disinfection device for grooming utensils according to an embodiment of the present invention, shown in use with a comb inserted into the device;

FIG. 3 is an elevated perspective view of the proximal end of the device shown in FIG. 2;

FIG. 4 is a plan view of the proximal end of the device shown in FIGS. 1 and 2;

FIG. 5 is an elevated perspective view of the device of in FIGS. 1 and 2, showing the distal end and ventilated wall portion;

FIG. 6 is an elevated perspective view of the device shown in FIGS. 1 and 2, showing the proximal end with an opening exposing the inner chamber with an inner sidewall arrangement with an array of LED elements;

FIG. 7 is an elevated perspective view of the inner sidewall assembly according to an embodiment of the device shown in FIG. 6, with an arrangement of receptacles in the sidewall for positioning an array of LED elements;

FIG. 8 is a cross-sectional view of the device shown in FIGS. 5 and 6, depicting the positioning of LED elements and areas of the housing for components of the device according to an embodiment;

FIG. 9 is a top plan view of a portable disinfection device for grooming utensils according to an embodiment of the present invention.

### DETAILED DESCRIPTION

The present invention as shown and described herein is a device, and method of using a device, which provides disinfection of hair grooming utensils and which is portable and easy to use, without risk of accidental spilling of chemicals or other problems associated with common products and methods. According to an embodiment, the present invention provides an apparatus and method which includes open access to an inner chamber for removable insertion of a portion of a utensil for treatment, without the need to open and close the housing when in use, and provides rapid treatment a high degree of efficacy for disinfecting the outers surface of the utensil.

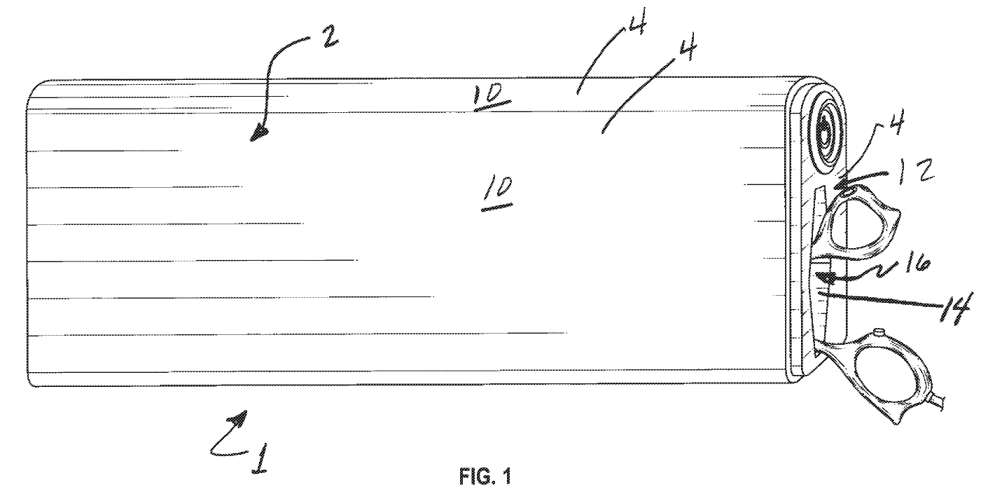

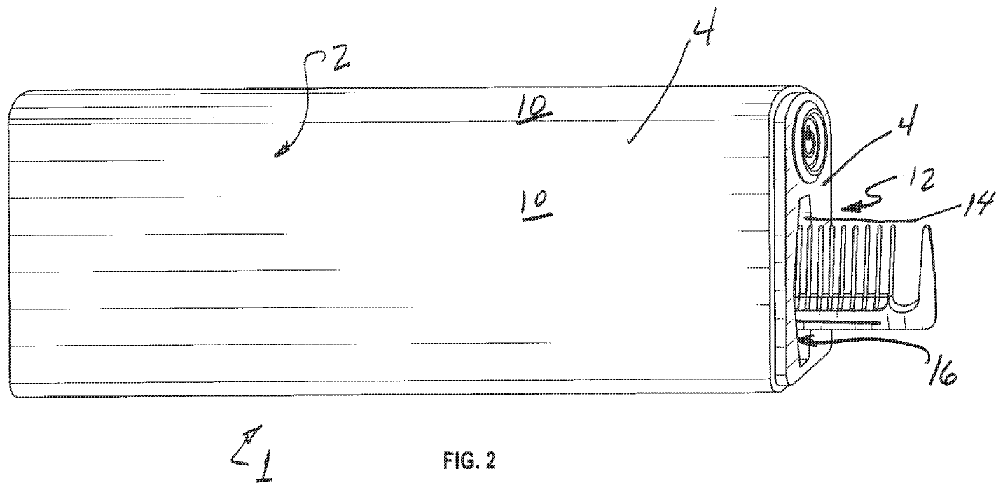

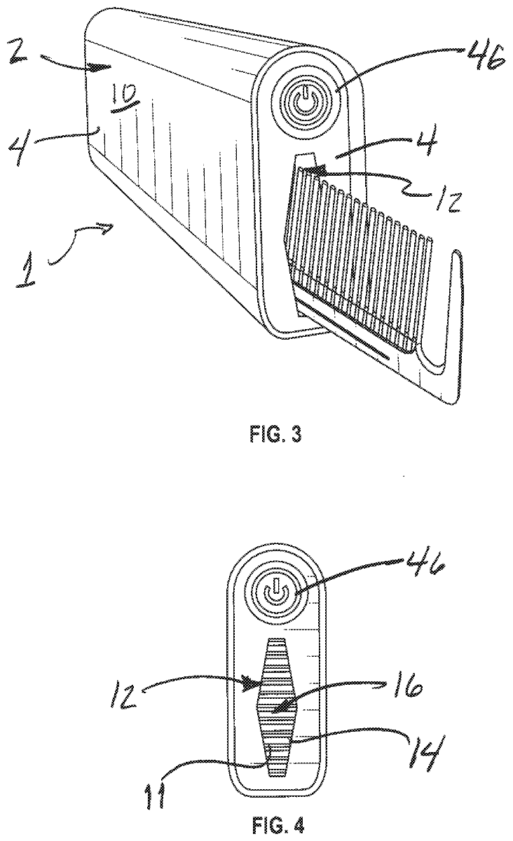

Specifically, as shown in FIGS. 1 and 2, a portable sanitation device 1 is provided for disinfecting hair grooming utensils, comprising a housing 2 formed of an outer sidewall arrangement 4 and having an inner sidewall arrangement 6 defining an inner chamber area 8. The housing 2 includes an opening 14 that is dimensioned to allow passage of a utensil into the inner chamber area 8, such that the utensil may be easily and rapidly removed from the inner chamber. As such, the opening 14 provides a passageway 16 for the utensil to be inserted into the elongated narrow inner chamber 8, and the utensil may be removed by simply tipping the device 1 slightly or by the user gripping a portion of the utensil that extends outward of the housing 2. In an embodiment, the housing 2 has an outer surface 10 with an access portion 12 or cover that is configured to be moved between a first position to expose the inner chamber 8 and a second position to cover the opening 14 that defines a passageway 16 into the inner chamber 8, thus at least partially covering or sealing the inner chamber 8.

According to an embodiment described herein and shown in the Figures, the opening 14 of the housing 2, preferably an elongated and generally rectangular shaped housing, is dimensioned to receive at least an extent of a hair grooming utensil. In an embodiment, the access portion 12 is an exposed opening at an end of the housing, preferably with the opening 14 dimensioned to substantially mate with the diameter dimensions of the inner sidewall 8, thereby at least substantially exposing the inner chamber 8 at the end and thus configured to provide a passageway 16 into the inner chamber 8. Alternatively, the access portion 12 may be a cover at an end portion of the housing 2 that may be moved, such as rotating the end portion about a hinge (not shown) joining the end portion to the wall of the housing 2. In an aspect of this embodiment, the hinged end portion may comprise a tethered or sliding wall segment such as a plug or cap, which may be moved relative the main body of the housing 2 to provide an opening 14 into the inner chamber 8. The opening 14, preferably located at a proximal end of the elongated body, is thus configured to permit an end portion of the utensil to protrude outward from the inner chamber 8 while the device is in use (i.e., when subjecting the utensil to treatment), so it may be easily grasped by a user to insert the utensil into, or remove the utensil from, the housing.

More specifically, the inner sidewall arrangement 6 of the device 1 preferably comprises at least two substantially opposed sidewalls 20, 22 that partially define the elongated inner chamber 8. This arrangement of the inner sidewalls 20, 22 provides generally opposed walls that are distanced apart to define a height of the inner chamber 8. In other words, the distance between the substantially opposed sidewalls 20, 22 at least partially defines the treatment area 24 of the inner chamber 8, which is a narrow elongated space dimensioned to receive an elongated extent of a hair grooming utensil, such the main body of a comb or the cutting portion of shears. When inserted into the housing, a second portion of the utensil, such as a gripping portion of a comb or the handle of shears, optionally may extend outward from the inner chamber 8, thus readily accessible for a user to grasp.

At least one ultra-violet emitting element 26, preferably a plurality of such elements, is positioned on the inner sidewall 6 surrounding the inner chamber 8, to emit ultra-violet light from generally opposite directions, thereby directing the emitted light toward the treatment area 24 of the inner chamber 8. More specifically, the plurality of elements 26 are preferably positioned on the at least two substantially opposed sidewalls (20, 22) along an extent of the inner chamber 8. In this arrangement, the elements 26 are configured to direct ultra-violet light toward the treatment area 24 of the inner chamber area 8, toward the entire outer surface of the portion of a utensil when inserted into the narrow elongated inner chamber 8. In an embodiment, an array of elements 26 are arranged along each of the substantially opposed sidewalls 20, 22, and preferably are arranged in at least two rows of elements 26 that each extend along a major extent of the length of the sidewalls 20, 22. In a preferred form of the embodiment, the elements 26 of each row are generally equally spaced from the other elements of the row, separated by spaces 27, and the elements 26 of a row are positioned adjacent the spaces 27.

In an embodiment such as is shown in FIG. 8, the array of elements 26 are positioned in an arrangement along at least two rows, with the elements 26 located in a staggered arrangement along the length of the elongated sidewalls 20, 22. The elements 26 residing along a first row are separated by spaces 27, and the elements 26 of a second row are positioned adjacent those spaces 27 along an extent of the sidewall length. This staggered arrangement of the array of elements 26 provides ultra-violet light emitting elements that are spaced in a desirable positions along the length of the inner sidewalls 20, 22 to provide evenly distribution of directed ultra-violet light emitted from the elements 26 toward the entire outer surface of a utensil residing within the inner chamber 8. In a preferred form of this embodiment, the inner chamber area 8 comprises an elongated space having cross-sectional dimensions with a height and width configured to receive an elongated extent of a grooming comb, and the light emitting elements 26 on the inner sidewalls 20, 22 are located less than about 2.0 inches, and preferably less than 1.5 inches, from a substantially opposed inner sidewall. In another aspect of this embodiment, the inner sidewall arrangement 6 surrounds an elongated inner chamber that has a width of less than about 3 inches, and preferably less than 2.5 inches, and a substantially smaller height, i.e., a height less than about 2.0 inches and preferably less than about 1.5 inches, and in a preferred embodiment less than about 1.0 inches. This arrangement of an elongated inner chamber 8 having a generally rectangular cross-sectional shape with elements 26 on opposing sidewalls 20, 22 positioned close together provides ultra-violet light emitted in close proximity to the outer surface of a utensil positioned within the inner chamber 8. The close proximity of the array of elements 26 surrounding the inner chamber 8, i.e., in close proximity to the central axis 9 (as defined by an axial line down the elongated inner chamber 8) provides high efficacy for sanitizing the utensil.

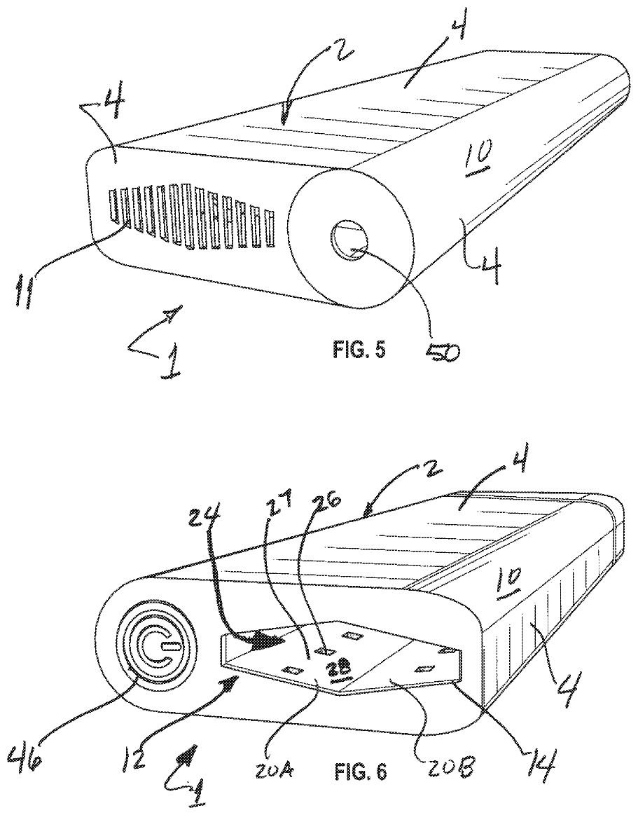

The housing 2 further includes a ventilated area 11, preferably located at a distal end located a distance from the proximal end having the opening 14. The ventilated area 11, such a screen or pattern of slots, is configured to provide passage of air between the inner chamber 8 and the area outside the housing 2. The ventilated area 11 of the housing 2 is configured to provide airflow through the housing 2 for dissipation of heat resulting from activation of the ultra-violet emitting elements 26, while also restricting or preventing the light from emitting from the inner chamber 8 and forming a blocking wall that encases a utensil inserted into the elongated inner chamber 8. In the embodiment shown in the Figures, such as FIG. 5, the ventilated area 11 includes an end panel of the housing 2 with slots configured to provide a plurality of passageways between the inner chamber 8 and the environment outside the device 1, while also providing a blocking component, or a stop, that positioned opposite the opening 14 for receiving a utensil into the inner chamber 8.

An extent, and preferably a major extent, of the elongated inner chamber 8 is preferably defined by reflective walls of the inner sidewall assembly, such as metal reflective surfaces 28 of the inner sidewall arrangement 6. Reflective surfaces 28 of the inner chamber 8 may be formed as generally opposing metal reflective surfaces 28 that are configured to reflect UV light from the emitting elements 26. In a preferred embodiment, the reflective surfaces 28 surrounding the inner chamber 8 include concave surfaces surrounding the treatment area 24 to reflect the UV light in alternate directions, and may include undulating curved surfaces to maximize directional variation of the UV light for treatment of a utensil in the treatment storage area 24. In another aspect of this embodiment, at least a portion of the inner wall arrangement 6 surrounding the inner chamber 8 is formed of sidewalls that are not entirely flat, thus minimizing flat surfaces and thereby reducing the areas in which the inner sidewall 6 is in contact with a utensil when placed in the storage area 24 of the inner chamber 8. Such curvature or curvilinear extent of the inner sidewall 6 provides the dual benefit of providing reflecting surfaces to angularly displace UV light toward the central region of the inner chamber 8, while also minimizing the surface area of the utensil in contact with the sidewall 6. Because such contact between the inner surface 6 and the outer surface of a utensil may result in lack of exposure to UV light, curved, curvilinear or undulating topography of the inner surface 6 provides increased exposure of the disinfecting treatment, while permitting a desirable amount of movement of the utensil within the storage area 24.

As shown an described, the inner sidewall arrangement 6 that defines at least an extent of the inner chamber area 8 may be formed as an elongated generally rectangular body comprised of substantially opposed sidewalls. In the embodiment shown in the Figures, the width of the inner chamber 8 is defined by substantially opposed sidewalls 20, 22 that hold the light emitting elements 26, and substantially shorter opposed sidewalls 23 residing along the height of the inner chamber 8. Mounting recesses or slots 25 are formed in the generally opposed side walls 20, 22 for positioning the light emitting elements 26. Commercially available strips with a row of LED elements (not shown) may be secured in place such that the elements 26 are easily positioned in rows on opposing side walls (20, 22), mating with the slots 25, thus forming one embodiment of an array of elements. Further, in the embodiment shown in the Figures, two rows of staggered arrangement of elements 26 are provided on each of the side walls. Specifically, as shown in FIG. 8, the rows of elements are staggered such that the elements 26 of one row are located adjacent the spaces 27 that reside between elements of the second row. This same staggered arrangement of elements is preferably located on each of the opposed sidewalls 20, 22, to optimize exposure of the ultra-violet light emitted into the inner chamber 8 on each side of a utensil inserted int the chamber 8.

In an embodiment, the elongated inner sidewall arrangement 6 is comprises generally opposed elongated sidewalls 20, 22, each being formed of two sidewall segments that are joined at an obtuse inner angle. More specifically, a first sidewall 20 is formed of two wall segments (20A and 20B) joined at an obtuse angle, and a second elongated sidewall 22 is formed of two wall segments (22A and 22B) joined at an obtuse angle. This arrangement of wall segments that form each sidewall 20, 22 is configured to provide sidewall segments that are directed toward each other, resulting in sidewalls 20, 22 of the inner chamber 8 being generally V-shaped in cross section rather than flat. In this arrangement, each sidewall provides mounting surfaces for the elements 26 in an angular position to direct the light emitting elements toward a central axis 9 of the inner chamber 8. In an embodiment as shown in the Figures, each sidewall 20, 22 has at least two rows of elements 26 arranged along a major extent of the length of the elongated inner chamber 8, wherein a row of elements is positioned on each of the wall segments (20A, 20B and 22A,22B), such that the elements 26 on each wall segment are generally directed toward the center of the inner chamber 8. This angular displacement of the elements 26 increases the effectiveness of the device for disinfecting the surface of a utensil that is inserted into the inner chamber 8. For example, positioning the elements in rows along adjacent elongated sidewall segments that are angled toward each other increases the overlap of light emitted from adjacent elements because the light emitting elements mounted on adjacent wall segments are directionally mounted to project ultra-violet light toward a central area of the inner chamber 8 to sanitize a utensil inserted into the inner chamber. Thus, having an internal structure in which the at least two substantially opposed elongated sidewalls are each formed of two elongated wall segments that are joined together at an obtuse angle to form a generally V-shaped elongated sidewall, the sidewall segments serve as mounting areas for the light emitting elements, wherein an array of elements are positioned along a major extent of the elongated V-Shaped wall segments and are thereby directed toward a central axis 9 of the inner chamber.

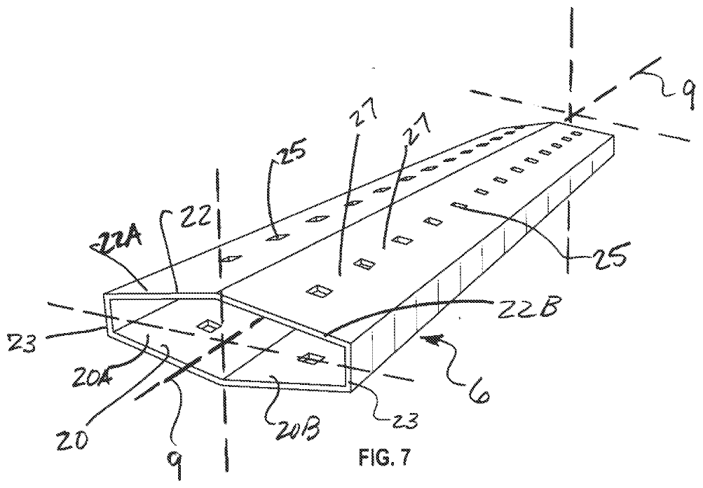

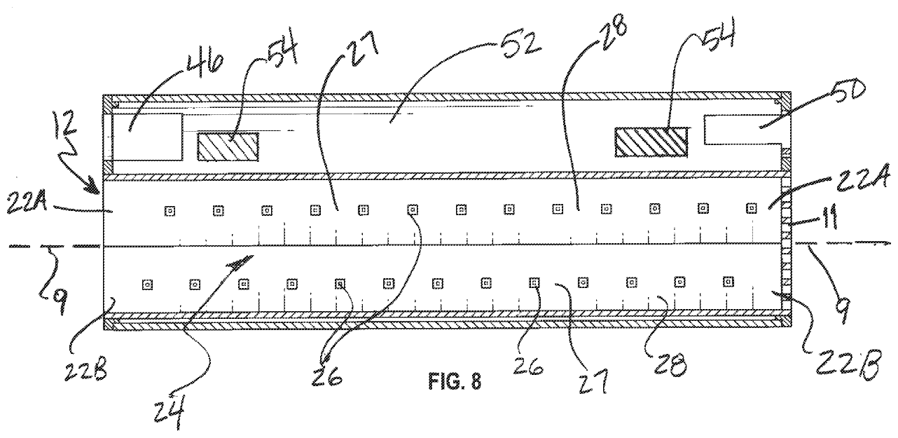

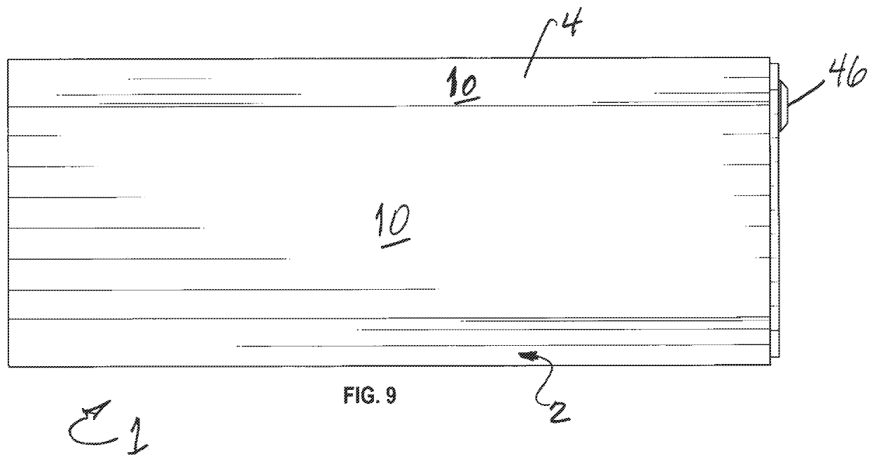

More specifically, such an arrangement of angularly displaced planar or curved sidewall segments provides a unified wall structure that surrounds the inner chamber 8, such that the ultra-violet light emitting elements 26 surround the inner chamber 26 and are generally directed toward a central axis 9 of the elongated inner chamber 8. In the embodiment shown in the Figures, such unified circumferential inner sidewall arrangement 6 is formed as a general oval arrangement of sidewall segments when viewed in cross section, as is depicted in the elevated perspective views of FIGS. 6 and 7. The general oval shape of the inner chamber 8 in the embodiment shown in the Figures is formed from as a hexagon of generally flat elongated wall segments. Specifically, the inner sidewall arrangement is formed from a pair of short sidewalls 23, preferably sidewalls approximately ¼ inch (0.25 inch) high, and at least two generally opposed wide sidewalls 20, 22 that are preferably about 2 inches wide, each comprised of two joined wall segments 20A, 20B and 22A, 22B. In an alternative embodiment, the substantially oval shape of the elongated inner sidewall arrangement may be comprised of curved sidewalls that cooperatively provide an oval shape with wall segments having different radius of curvature when viewed in cross section.

A control module is located within the housing 2, electrically connected to the elements 26, and is configured to selectively activate the element 26 to emit ultra-violet light directed toward the inner chamber 8. Such activation may be through a control module comprising a processor and memory that is configured to selectively activate the treatment assembly 26 for a determined amount of time. In such an embodiment, the control module may include a processor module and a memory module, wherein the control module is programed to activate the elements 26 for a period of time based on a timer function of the processor. The time duration of activation of the UV light, thus length of time of exposure of the sanitizing treatment, is preferably predetermined and may be programed into the memory, and/or may be selectable by a user depending on the type or size of the utensil.

In an embodiment, the control module may also be associated with a detector for determining when the access portion or cover of the housing is in an open or closed position, wherein the control module is prevented from activating the element(s) 26 when the access portion, or cover, is in the open or closed position. In one example of this embodiment, the control module receives a signal from a sensor indicating position of an end cap that is moveable between a first and second position, and the control module prevents activation when undesirable. In one embodiment, activation of the elements is prevented and/or terminated when the cover or end cap is in the closed position, i.e., activation is prevented when a utensil cannot be inserted into the housing for treatment. In another embodiment, activation of the elements 26 is prevented when the opening is not covered by a protective shield preventing ultra-violet rays from emitting from the opening (such as a translucent or transparent cover configured to substantially block such wavelength(s) of light. Thus, the device 1 may include mechanical circuitry, in combination with programing of a processor, for the control module to receive a signal indicating the position or state of the end cap or a cover over the opening, to selectively activate the elements 26.

In a preferred embodiment, at least a portion of the inner wall 6 of the device, and more specifically the treatment area 24 for disinfecting a utensil, includes a metal reflective surface that is configured to reflect the ultra violet emission from the elements. Such reflective surface preferably comprises a concave region located at, and at least partially surrounds, the treatment, or storage, area 24 of the inner chamber 8. This arrangement provides a reflective surface 28 surrounding the treatment area of the inner chamber so the ultra-violet light emitting from the element(s) 26 is simultaneously applied to multiple surfaces of the utensil when the elements 26 are activated. This feature minimizes or avoids the need to change orientation or placement of the utensil within the inner chamber 8 during treatment. In another aspect of a preferred embodiment, the inner walls of the device, at least in the storage/treatment area 24 of the inner chamber 8, has curved shape, or a V-shape of wall segments. This aspect of the invention, which provides preferred directed mounting of the elements 26 surrounding the inner chamber 8 as described herein, also provides one or more concave surfaces of the inner wall to increase exposure of the outer surface of the utensil when placed in the storage area 24 for treatment. Having at least an extent of the inner surface concave (such as by the configuration of curved or curvilinear the inner sidewalls 6) reduces the contact area between the inner wall and the outer surface of the utensil. In a preferred embodiment, the inner chamber has a characteristic width defined by the inner sidewall that is about 2 inches wide, and has a length at least about 9 inches long. Such dimensions of the inner sidewall arrangement, and resulting dimensions of the inner chamber 8, is configured to accommodate typical maximum dimensions of the extent of utensils to be disinfected, such as the toothed body of a hair comb that is usually less than 2.0 inches wide, or the cutting portion of shears that are commonly less than 9 inches in length, while providing a structure for mounting elements 26 in close proximity to the treatment area 24 of the inner chamber 26.

In an embodiment, a power source is provided, preferably including a mode of operation in which the power source is a rechargeable battery which resides in a portion of the housing between an outer wall 4 an inner wall 6. The battery is electrically connected to the UV light emitting elements 26 and the control module. In one embodiment, this connection includes connection directly to the elements 26. In another embodiment, the electrical connection includes switches and/or controls, such as battery connection to the control module and/or a switch actuator which may be controlled by a user, which then transmits electrical charge to the UV light elements 26. A power connection receptacle 50 is provided on the housing 2, such as a common low voltage USB type connection, for supplying power to the device and to supply electrical charge to the battery. A user interface or actuator 46 is also provided, which may include a mechanical button or switch, dial, slide controller, or similar functional mechanism for a user to send signal for functions of the device 1. In a preferred embodiment, the user interface (actuator 46) may be a button with an indicator light, wherein a user simply pushes the button to activate functions of the device. For example, a user may push the button to signal activation of the UV light elements. In a form of the invention, an indicator light emits a color indicating activation of the elements (such as the color red) and the control module changes the indicator light to a different color (such as green) when a treatment cycle is complete.

It is understood that other mechanisms may be employed for controlling the device of the present invention, including wireless connectivity to a remote device, such as a cell phone or a wireless controller utilizing program control strings. Further, power to the device and to the battery may optionally be provided by direct electrical wiring from a power source, or by a solar power generating assembly such as a solar power panel residing on a portion of the outer wall of the housing of the device. In the embodiment shown in FIG. 8, the housing 2 includes an auxiliary compartment 52 adjacent the inner sidewall arrangement 6 that forms walls of the inner chamber 8. The auxiliary compartment 52, which is adjacent the power actuator 46 and the power receptacle 50, is configured to provide a suitable mounting position for a battery (not shown) as well as operation components such as the control module, a memory module, processor and orientation sensor if desired. In a preferred embodiment, such as shown in the Figures, the axillary compartment is an elongated space that extends along the length of the inner chamber 8 adjacent the inner sidewall arrangement 6, and preferably extends between the power actuator 46 and the power receptacle 50.

In an embodiment, a magnetic mounting assembly 54 is located within the housing 2 inside the outer sidewall arrangement 4. The magnet assembly 54 preferably includes an elongated magnet arrangement extending along a major extent of the length of the elongated housing 2, either as a single elongated magnet or least two magnets positioned within the outer sidewall arrangement 4 of the housing 2, located adjacent the inner sidewall arrangement 6. The magnet mounting assembly 54 within the housing 2 is configured to align with one or more mating surfaces for magnetic connection of the housing to a surface, to secure the device 1 in position where desired. In one example as shown in FIG. 8, the magnetic mounting assembly 54 comprises at least two magnets located within the auxiliary compartment 52 of the housing 2 wherein the magnets of the assembly 54 are spaced apart along an extent of the length of the elongated housing 2. This arrangement 54 preferably is configured to mate with similarly spaced magnets on a mounting plate (not shown) that is secured to a surface at a salon, such as a horizontal surface on the side of a cabinet or under a shelf. In this configuration, the mounting assembly 54 is used to secure the device 1 in position and is easily removed or tilted (by disengaging one of the magnets from its mating magnet on the mounting plate) to tilt the device for removing a comb or a similar utensil from within the inner chamber 8.

In an embodiment of the portable disinfecting device, the plurality of UV light emitting elements 26 include LED elements. Preferably, a plurality of LED diodes are arranged along an a major extent along generally opposed sides of the inner chamber 8. In examples shown in the Figures, the elongated body of the device includes an inner chamber that is approximately nine (9) inches long, with twenty-six (26) UV light emitting LED elements arranged along each of generally opposed sidewalls of the sidewall arrangement 6 forming the inner chamber 8. In this embodiment, the LED elements are substantially equally spaced along the length of the treatment area 24 of the inner chamber 8. Depending on the length of the elongated housing 2 and the intended treatment area 24 of the inner chamber 26, at least three (3) to ten (10) UV emitting elements 26 are equally spaced along each side of the inner chamber 8. In a preferred embodiment as shown in FIG. 8, approximately twenty-six (26) LED elements are arranged along each of two generally opposed sides of the inner sidewall arrangement, wherein the inner chamber is approximately 8-10 inches in length. The LED elements are preferably arranged in pairs along the opposing side walls for even distribution of the ultra-violet emission throughout the length of the inner chamber 8. The elements 26 are selectively activated by an actuator 46, either by direct mechanical activation or through the control module. In a preferred embodiment, the arrangement of elements 26 on each sidewall of the inner chamber 8 are activated simultaneously, thereby subjecting the entire outer surface of the utensil (i.e., the extent of the utensil inserted into the inner chamber) for treatment. In this embodiment, the reflective surfaces 28 of the inner sidewall 6 that defines the inner chamber 8 are preferably formed of aluminum or similar metal suitable to reflect UV light wavelength, to facilitate exposure to UV light in multiple directions within the inner chamber 8 toward the utensil.

A method is also provided, in which a hair grooming utensil is subjected to disinfection treatment with portability and ease of access of the utensil being treated. The method includes providing a portable device having a housing 2 with an inner chamber 8 configured to receive a hair grooming utensil within a treatment area 24 of the chamber. At least one ultra-violet emitting element 26, and preferably a plurality of elements, positioned within the inner chamber 8 are selectively activated to emit ultra-violet light toward a grooming utensil inserted into the inner chamber 8. In a preferred form of the method, an array of ultra-violet emitting elements is provided on each of two generally opposed sidewalls surrounding the inner chamber 8, with the elements in close proximity of the outer surface of a grooming utensil inserted into the inner chamber. This close proximity is achieved by providing a device having the elements of the generally opposed sidewalls less than about 1.5 inches from each other, and preferably no greater than about 1.0 inch apart. The method includes the step of activating the element(s) 26 by an actuator 46 and/or a control module to cause the elements 26 to emit ultra violet light for sanitizing the outer surface of the utensil. According to this method, an extent of the utensil is inserted into the inner chamber 8 for treatment, by inserting a portion of the utensil through an open end of the housing 2. The opening allows for the utensil to be easily removed, such as a handle of the utensil extending from the housing or a comb sliding out of the inner chamber 8 by a user simply tilting the device. Further, in an embodiment in which the housing comprises a hinged or removable end segment, the method includes a step of moving the end segment of the housing 2 to expose the opening, and then inserting at least an extent of the utensil into the inner chamber 8.

The step of activating the elements 26 preferably includes activating at least two elements 26 that are positioned on opposite sides of the inner chamber 8 to simultaneously emit ultra violet light toward opposite sides of the utensil within inner chamber 8. In a preferred form of the invention, as is described in further detail herein, a plurality of elements 26 are positioned along opposite sides of the inner chamber 8, and the method includes simultaneous activation of the elements on each side of the inner chamber. Further, the method may include activating elements 26 for a predetermined duration, such as by employing a timer function of the control module. In an alternate embodiment, the control module utilizes information from an orientation sensor whereby emission of ultra violet light is prolonged until a determination is made by the control module that all surfaces of a utensil within the storage area have been exposed to disinfecting treatment. As yet another alterative embodiment, of the method, ultra violet light is emitted from the elements 26 for a predetermined amount of time prior to, and after, the device has been moved, such that all surfaces of the utensil have been exposed to the UV light treatment within the storage area. Thus, as depicted in FIG. 11, the method includes at least a first cycle and a second cycle of sanitizing exposure, each for a predetermined minimum duration of time, separated by an indication of movement of the device and/or completely turning over the device.

What is claimed is:

1. A portable sanitation device for sanitizing treatment of hair grooming utensils, comprising:
a housing having an outer sidewall arrangement and an inner sidewall arrangement partially surrounding an elongated inner chamber region, said housing having an opening formed in said outer sidewall and said inner sidewall arrangement, said opening being configured to allow passage of a grooming utensil into said elongated inner chamber region;
said inner sidewall arrangement comprising at least two substantially opposed elongated sidewalls, a plurality of ultra-violet light emitting elements positioned on each of said at least two substantially opposed sidewalls, said elements being configured to direct ultra-violet light toward the inner chamber.

2. The portable sanitation device of claim 1, wherein;
said plurality of light emitting elements comprises an array of elements, the elements of each array being evenly spaced along the elongated length of each of said substantially opposed sidewalls to direct light along a length of the inner chamber.

3. The portable sanitation device of claim 2, wherein;
said array of elements arranged along said sidewalls includes at least two rows of elements extending along each of said substantially opposed sidewalls.

4. The portable sanitation device of claim 3, wherein;
said elements positioned along at least two rows are located in a staggered arrangement along the length of the sidewall, such that elements residing along a first row are separated by spaces and the elements of a second row are positioned adjacent said spaces.

5. The portable sanitation device of claim 1, wherein;
said inner chamber comprises an elongated space having a height and width configured to receive an elongated grooming comb, and said light emitting elements of said inner sidewall arrangement are located less than about 1.5 inches from a substantially opposed inner sidewall.

6. The portable sanitation device of claim 2, wherein;
said at least two substantially opposed elongated sidewalls are each formed of two elongated wall segments joined together at an obtuse angle to form a generally V-shaped elongated sidewall, wherein said array of elements are positioned along a major extent of the elongated V-Shaped wall segments such that the array of elements are directed toward a central axis of the inner chamber.

7. The portable sanitation device of claim 1, wherein;
said inner chamber comprises an elongated space having a height and width configured to receive an elongated grooming comb, and said light emitting elements on said inner sidewall arrangement are located less than about 0.5 inches from the grooming comb when inserted into the inner chamber.

8. The portable sanitation device of claim 1, wherein;
at least a portion of said inner sidewall is formed of opposed generally concave metal surfaces.

9. The portable sanitation device of claim 1, wherein;
said inner sidewall comprises reflective surface configured to reflect the ultra violet light emission from said light emitting elements, at least an extent of said reflective surface comprising a generally concave region.

10. The portable sanitation device of claim 1, wherein;
said opening of the housing is comprised of a void area of the outer wall at an end of the housing, said void area being dimensioned as a passageway into said inner chamber and configured to allow passage of an extent of an elongated grooming utensil such that a portion of the utensil resides within the inner chamber and a portion of the utensil extends radially outward of the housing.

11. The portable sanitation device of claim 1 wherein;
said access portion is comprised of an opening of the housing, said opening is aligned with the inner sidewall arrangement and has a width that is less than about 2.5 inches and a height of less than about 1.0 inch.

12. The portable sanitation device of claim 1, wherein;
said housing comprising an access portion configured to be moved between a first position to cover said opening to the inner chamber area and a second position to expose said opening.

13. The portable sanitation device of claim 12, wherein;
said access portion comprises a hinged end cap that is rotatable about a hinged connection between said first and said second position.

14. The portable sanitation device of claim 1, further comprising;
a rechargeable battery positioned adjacent between the outer sidewall arrangement and said inner sidewall, said battery being electrically connected to a control module configured to selectively activate said plurality of elements.

15. A method for subjecting a hair grooming utensil to disinfection treatment, comprising:
providing a portable device having a housing with an elongated inner chamber and an end opening, said opening being configured to provide a passageway for receiving an extent of a hair grooming utensil within said inner chamber, wherein a plurality of ultra-violet light emitting elements are positioned along said inner chamber and selectively activated to emit ultra-violet light toward said portion of the grooming utensil within the inner chamber;
inserting a utensil into the passageway such that the utensil resides within the inner chamber in close proximity to said elements;
activating said elements with a control module, to cause said elements to emit ultra violet light toward said utensil residing within said inner chamber, such that the entire outer surface of said portion of the utensil is subjected to ultra violet light from the elements; and, after activating said elements for a defined period of time, removing said utensil from the housing through said opening.

16. The method of claim 15, wherein said step of activating the elements includes the step of activating at least two elongated rows of elements, each said row of elements residing on substantially opposing sides of the inner sidewalls to simultaneously emit ultra violet light toward opposite sides of the utensil.

17. The method of claim 15 wherein the step of inserting the utensil into the inner chamber includes the step of inserting a portion of the utensil into the inner chamber region and a portion of the utensil extends through said opening and radially outward of the housing.

18. A portable device for sanitizing treatment of hair grooming utensils, comprising:
a housing formed of an outer sidewall arrangement and an elongated inner sidewall arrangement within the housing with an inner chamber residing between the sidewall arrangement;
an opening of the housing and inner sidewall located at a first end of the portable device, said opening configured to provide a passageway into said inner chamber from outside the housing, wherein said opening is dimensioned to receive an elongated portion of a hair grooming utensil within the inner chamber;
an array of light emitting elements configured to direct ultra-violet light toward the inner chamber to disinfect surfaces of a utensil residing within the inner chamber, said array comprising elements positioned on at least two substantially opposed inner sidewalls positioned less than 2.0 inches apart, wherein said elements of a first said substantially opposed sidewalls is configured to emit ultra-violet light toward a second of said substantially opposed sidewalls.

19. The portable device of claim 18, wherein said opening is configured to provide a passageway for a portion of an elongated utensil body extending into the inner chamber and a second portion of the utensil body extending outward from the housing, such that said array of light emitting elements is configured to direct ultra violet light toward the outer surface of the utensil body residing within the inner chamber.

20. The portable device of claim 18, wherein said housing includes a ventilated grill portion and an end portion comprising a cover moveable between an open position in which the opening to the inner chamber is exposed and a closed position in which the opening of the inner chamber is at least substantially covered.
